(19) United States
(12) Patent Application Publication
(10) Pub. No.: US 2014/0371844 A1
(43) Pub. Date: Dec. 18, 2014

(54) TRANSCATHETER MITRAL VALVE AND DELIVERY SYSTEM

(71) Applicant: St. Jude Medical, Cardiology Division, Inc., St. Paul, MN (US)

(72) Inventors: Theodore Paul Dale, Corcoran, MN (US); Mathias C. Glimsdale, St. Michael, MN (US); Mark Krans, Hopkins, MN (US)

(21) Appl. No.: 14/306,356

(22) Filed: Jun. 17, 2014

Related U.S. Application Data
(60) Provisional application No. 61/969,445, filed on Mar. 24, 2014.
(60) Provisional application No. 61/836,427, filed on Jun. 18, 2013.

(51) Int. Cl. A61F 2/24

(52) U.S. Cl. CPC A61F 2/2418; A61F 2/2427
USPC 623/2.11; 623/2.18

(57) ABSTRACT

A prosthetic heart valve includes a collapsible and expandable stent, a collapsible and expandable valve assembly disposed within the stent, and one or more features coupled to the stent for at least partially anchoring the prosthetic heart valve within a native valve annulus of a patient. The anchoring features may include a body transitionable from an unfurled condition to a furled condition, the furled condition forming a flange for at least partially anchoring the heart valve, and/or a plurality of hooks transitionable from a deformed condition to a relaxed condition for at least partially anchoring the heart valve. A delivery device for the prosthetic heart valve may include a handle and a catheter member extending from the handle and having a first portion, a second portion, and a compartment for receiving the valve. The delivery device may provide for staged deployment of the valve.

### CROSS-REFERENCE TO RELATED APPLICATIONS

The present application claims the benefit of the filing dates of U.S. Provisional Patent Application No. 61/836,427, filed Jun. 18, 2013 and titled “ANCHORED MITRAL VALVE PROSTHESIS,” and U.S. Provisional Patent Application No. 61/969,445, filed Mar. 24, 2014 and titled “TRANSCATHETER MITRAL VALVE AND DELIVERY SYSTEM,” the disclosures of which are both hereby incorporated by reference herein.

### BACKGROUND OF INVENTION

The present disclosure relates to heart valve replacement and, in particular, to the delivery of collapsible prosthetic heart valves. More particularly, the present disclosure relates to devices and methods for delivering collapsible prosthetic heart valves within native valve annuluses.

Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible prosthetic heart valves typically take the form of a valve structure mounted on a stent. There are two types of stents on which the valve structures are ordinarily mounted: a self-expanding stent and a balloon-expandable stent. To place such valves into a delivery apparatus and ultimately into a patient, the valve is generally first collapsed or crimped to reduce its circumferential size.

When a collapsed prosthetic valve has reached the desired implant site in the patient (e.g., at or near the annulus of the patient's heart valve that is to be replaced by the prosthetic valve), the prosthetic valve can be deployed or released from the delivery apparatus and re-expanded to full operating size. For balloon-expandable valves, this generally involves releasing the entire valve, assuring its proper location, and then expanding a balloon positioned within the valve stent. For self-expanding valves, on the other hand, the stent automatically expands as the sheath covering the valve is withdrawn.

### SUMMARY OF THE INVENTION

In some embodiments, a method of deploying a prosthetic heart valve from a delivery device at a target site in a patient includes introducing the prosthetic heart valve to the target site in a collapsed configuration, transitioning a plurality of hooks from a deformed condition to a relaxed condition, transitioning a body from an unfurled condition to a furled condition and decoupling the prosthetic heart valve from the delivery device after the plurality of hooks are in the relaxed condition and the body is in the furled condition, whereby the plurality of hooks and the body cooperate to anchor the prosthetic heart valve at the target site. The target site may be the mitral valve annulus of the patient.

In some embodiments, a prosthetic heart valve having an inflow end and an outflow end may include a stent having a collapsed condition and an expanded condition, a collapsible and expandable valve assembly disposed within the stent and having a plurality of leaflets, and a plurality of hooks coupled to the stent and transitionable between a deformed condition and a relaxed condition, the plurality of hooks extending toward the inflow end in the relaxed condition to at least partially anchor the prosthetic heart valve within a native valve annulus.

In other embodiments, a prosthetic heart valve having an inflow end and an outflow end includes a stent having a collapsed condition and an expanded condition, a collapsible and expandable valve assembly disposed within the stent and having a plurality of leaflets and a body coupled to the stent and formed of braided wire, the body being transitionable between an unfurled condition and a furled condition, the furled condition forming a flange capable of at least partially anchoring the prosthetic heart valve within a native valve annulus.

In yet other embodiments, a method of deploying a prosthetic heart valve from a delivery device at a target site in a patient, the heart valve including a stent having a collapsed condition and an expanded condition, a collapsible and expandable valve assembly disposed within the stent, a body assembled to the stent and transitionable between a furled condition and an unfurled condition, and a plurality of hooks coupled to the stent and being transitionable between a deformed condition and a relaxed condition, may include (i) introducing the prosthetic heart valve to the target site in a collapsed condition; (ii) deploying the plurality of hooks to transition the plurality of hooks from the deformed condition to the relaxed condition; (iii) deploying the body to transition the body from the unfurled condition to the furled condition; and (iv) decoupling the prosthetic heart valve from the delivery device after the plurality of hooks are in the relaxed condition and the body is in the furled condition, whereby the plurality of hooks and the body cooperate to anchor the prosthetic heart valve at the target site.

In still other embodiments, a delivery device for a collapsible medical device may include a handle and a catheter member extending from the handle and having a first portion, a second portion, and a compartment for receiving the medical device, the first portion being operably coupled to a first shaft that is axially translatable with respect to the handle and the second portion being operably coupled to a second shaft that is axially translatable with respect to the handle and to the first shaft.

In further embodiments, a method of delivering a medical device into a patient may include (a) providing a delivery device including a handle and a catheter member extending from the handle and having a first portion with a first shaft operably coupled thereto, a second portion with a second shaft operably coupled thereto, and a compartment, the medical device being positioned in the compartment; (b) advancing the catheter member to an implant site within the patient; (c) axially translating the second shaft in a first axial direction; (d) axially translating the first shaft in a second axial direction opposite the first axial direction; and (e) further axially translating the second shaft in the first axial direction; whereby each axial translation in steps (c) through (e) is performed in sequence and each sequential axial translation at least partially releases the medical device from the compartment.

### BRIEF DESCRIPTION OF THE DRAWINGS

Various embodiments of the present disclosure are disclosed herein with reference to the drawings, wherein:

FIG. 1 is a schematic cutaway representation of a human heart showing a transapical delivery approach;

FIG. 2 is a schematic representation of a native mitral valve and associated cardiac structures;

FIG. 3A is a longitudinal cross-section of one embodiment of a prosthetic heart valve having a stent, a valve assembly and a frame, the valve being in a relaxed configuration;

FIG. 3B is a longitudinal cross-section of the prosthetic heart valve of FIG. 3A in a constrained configuration;

FIG. 3C is an enlarged partial side view of the prosthetic heart valve of FIG. 3A in a relaxed configuration;

FIG. 3D is a partial schematic representation of the prosthetic heart valve of FIG. 3A disposed in a native valve annulus;

FIG. 4A is a longitudinal cross-section of another embodiment of a prosthetic heart valve having a stent, a valve assembly and anchoring hooks, the valve being in a relaxed configuration;

FIG. 4B is a longitudinal cross-section of the prosthetic heart valve of FIG. 4A in a constrained configuration;

FIG. 4C is an enlarged partial side view of the prosthetic heart valve of FIG. 4A in a relaxed configuration;

FIG. 4D is a partial schematic representation of the prosthetic heart valve of FIG. 4A disposed in a native valve annulus;

FIG. 5A is a longitudinal cross-section of yet another embodiment of a prosthetic heart valve having a stent, a valve assembly, a flange and anchoring hooks, the valve being in a relaxed configuration;

FIG. 5B is a longitudinal cross-section of the prosthetic heart valve of FIG. 5A in a constrained configuration;

FIG. 5C is partial schematic representation of the prosthetic heart valve of FIG. 5A disposed in native valve annulus;

FIG. 5D is an enlarged partial side view of a prosthetic heart valve in a relaxed configuration according to an aspect of the disclosure;

FIGS. 6A and 6B are schematic top views of prosthetic heart valves having a circular transverse cross-section and a D-shaped transverse cross-section, respectively;

FIG. 7 is a perspective view of a delivery device with an outer sheath being shown as partially transparent;

FIG. 8 is a partially exploded view of a handle subassembly and an actuator subassembly of the delivery device of FIG. 7;

FIG. 9 is a perspective view of the delivery device of FIG. 7 with a portion of the handle subassembly removed;

FIG. 10A is a perspective view of a distal portion of the delivery device of FIG. 7 shown as partially transparent;

FIG. 10B is a schematic cross-sectional representation of a distal portion of the delivery device of FIG. 7; and

FIGS. 11A-I are sequential schematic side views showing the deployment of a prosthetic heart valve using the delivery device of FIG. 7.

Various embodiments of the present disclosure will now be described with reference to the appended drawings. It is to be appreciated that these drawings depict only some embodiments of the disclosure and are therefore not to be considered limiting of its scope.

### DETAILED DESCRIPTION

Despite the various improvements that have been made to collapsible prosthetic heart valves and delivery systems, conventional devices, systems, and methods suffer from some shortcomings. In conventional collapsible heart valves, the stent is usually anchored within the native valve annulus via the radial force exerted by the expanding stent against the native valve annulus. If the radial force is too high, damage may occur to heart tissue. If, instead, the radial force is too low, the heart valve may move from its implanted position, for example, into either the left ventricle or the left atrium, requiring emergency surgery to remove the displaced valve. Because this radial force anchoring partly depends on the presence of calcification or plaque in the native valve annulus, it may be difficult to properly anchor the valve in locations where plaque is lacking (e.g., the mitral valve annulus). Moreover, in certain applications, such as mitral valve replacement, the heart valve may require a lower profile so as not to interfere with surrounding tissue structures. Such a low profile makes it difficult for the valve to remain in place.

In view of the foregoing, there is a need for further improvements to the devices, systems, and methods for prosthetic heart valve implantation and the anchoring of collapsible prosthetic heart valves, and in particular, self-expanding prosthetic heart valves. Among other advantages, the devices, systems and methods of the present disclosure may address one or more of these needs.

Blood flows through the mitral valve from the left atrium to the left ventricle. As used herein, the term “inflow end,” when used in connection with a prosthetic mitral heart valve, refers to the end of the heart valve closest to the left atrium when the heart valve is implanted in a patient, whereas the term “outflow end,” when used in connection with a prosthetic mitral heart valve, refers to the end of the heart valve closest to the left ventricle when the heart valve is implanted in a patient. Further, when used herein with reference to a delivery device, the terms “proximal” and “distal” are to be taken as relative to a user using the device in an intended manner. “Proximal” is to be understood as relatively close to the user and “distal” is to be understood as relatively farther away from the user. Also, as used herein, the terms “substantially,” “generally,” and “about” are intended to mean that slight deviations from absolute are included within the scope of the term so modified.

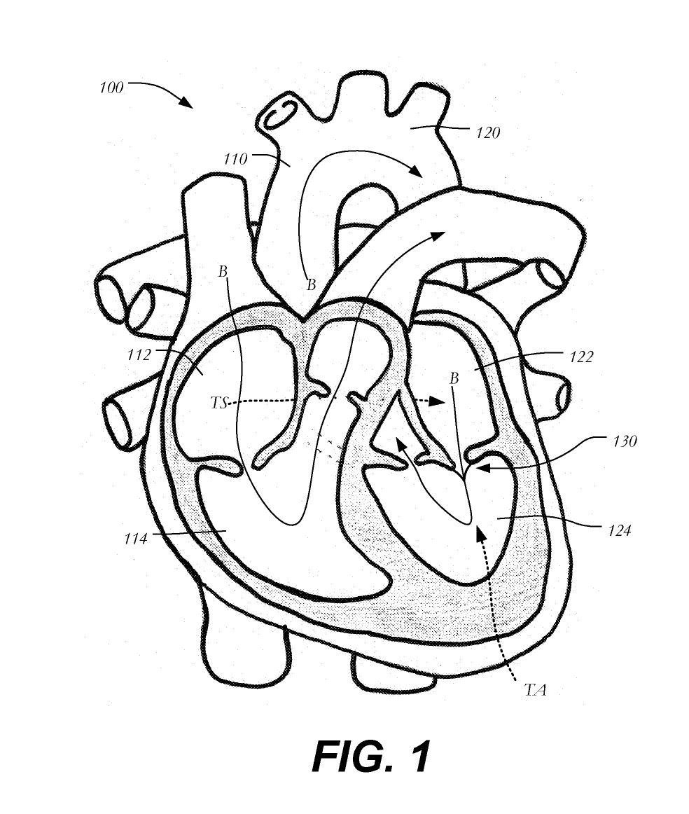

FIG. 1 is a schematic cutaway representation of human heart 100. The human heart includes two atria and two ventricles: right atrium 112 and left atrium 122, and right ventricle 114 and left ventricle 124. Heart 100 further includes aorta 110, and aortic arch 120. Disposed between the left atrium and the left ventricle is mitral valve 130. Mitral valve 130, also known as the bicuspid valve or left atrioventricular valve, is a dual-flap that opens as a result of increased pressure in left atrium 122 as it fills with blood. As atrial pressure increases above that of left ventricle 124, mitral valve 130 opens and blood passes into left ventricle 124. Blood flows through heart 100 in the direction shown by arrows “B”.

A dashed arrow, labeled “TA”, indicates a transapical approach of implanting a prosthetic heart valve, in this case to replace the mitral valve. In transapical delivery, a small incision is made between the ribs and into the apex of left ventricle 124 to deliver the prosthetic heart valve to the target site. A second dashed arrow, labeled “TS”, indicates a transeptal approach of implanting a prosthetic heart valve where the valve is passed from right atrium 112 to left atrium 122. Other approaches for implanting a prosthetic heart valve are also possible.

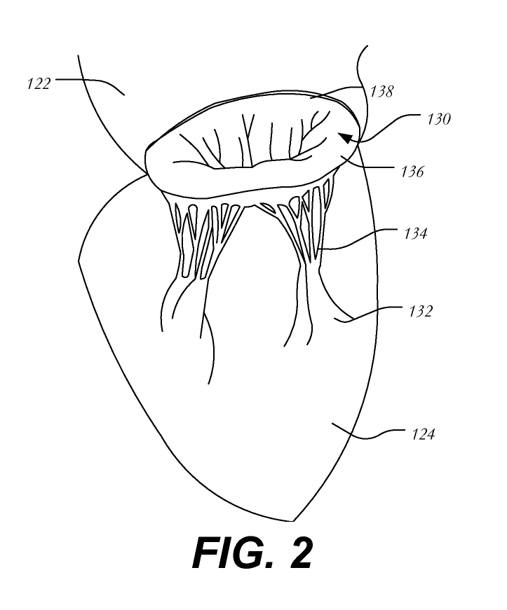

FIG. 2 is a more detailed schematic representation of native mitral valve 130 and its associated structures. As previously noted, mitral valve 130 includes two flaps or leaflets, posterior leaflet 136 and anterior leaflet 138, disposed between left atrium 122 and left ventricle 124. Cord-like tendons, known as chordae tendineae 134, connect the two leaflets 136, 138 to the medial and lateral papillary muscles 132. During atrial systole, blood flows from higher pressure in left atrium 122 to lower pressure in left ventricle 124. When left ventricle 124 contracts in ventricular systole, the increased blood pressure in the chamber pushes leaflets 136, 138 to close, preventing the backflow of blood into left atrium 122. Since the blood pressure in left atrium 122 is much lower than that in left ventricle 124, leaflets 136, 138 attempt to evert to the low pressure regions. Chordae tendineae 134 prevent the eversion by becoming tense, thus pulling on leaflets 136, 138 and holding them in the closed position.

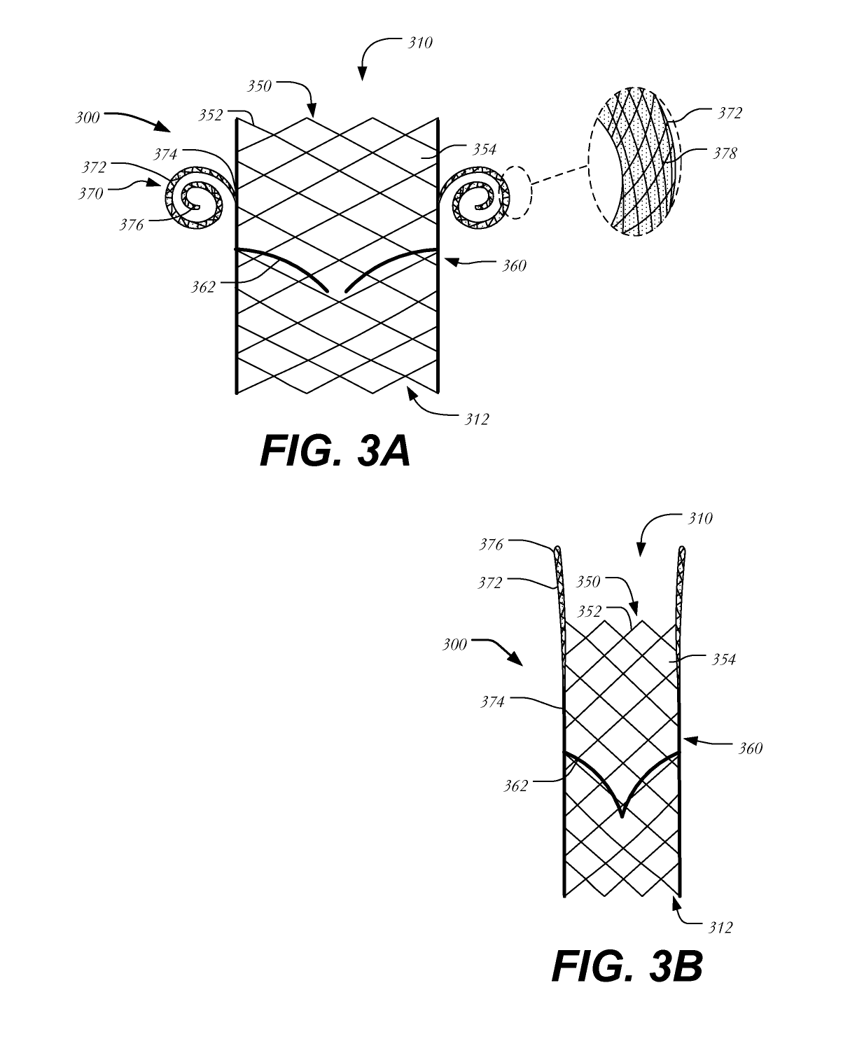

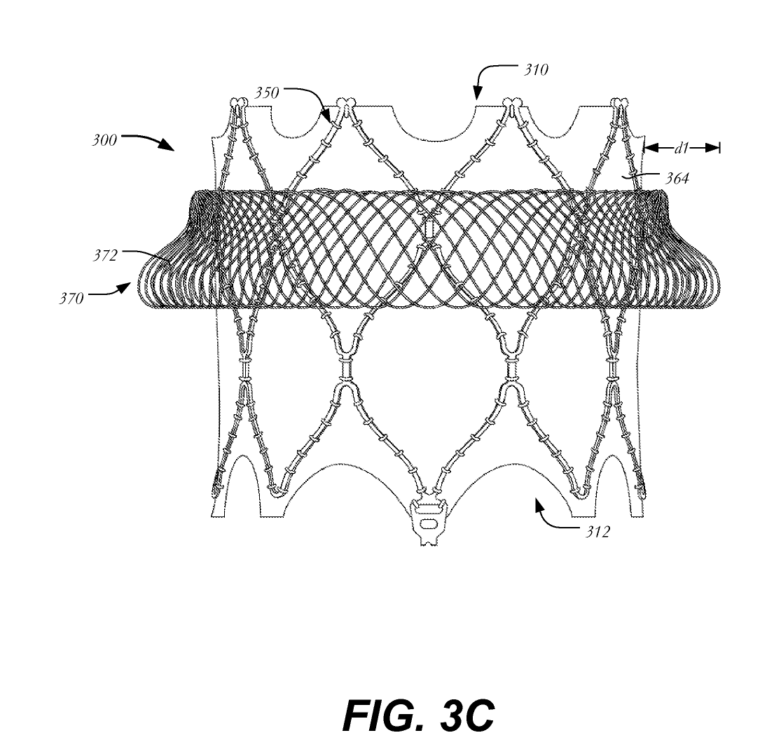

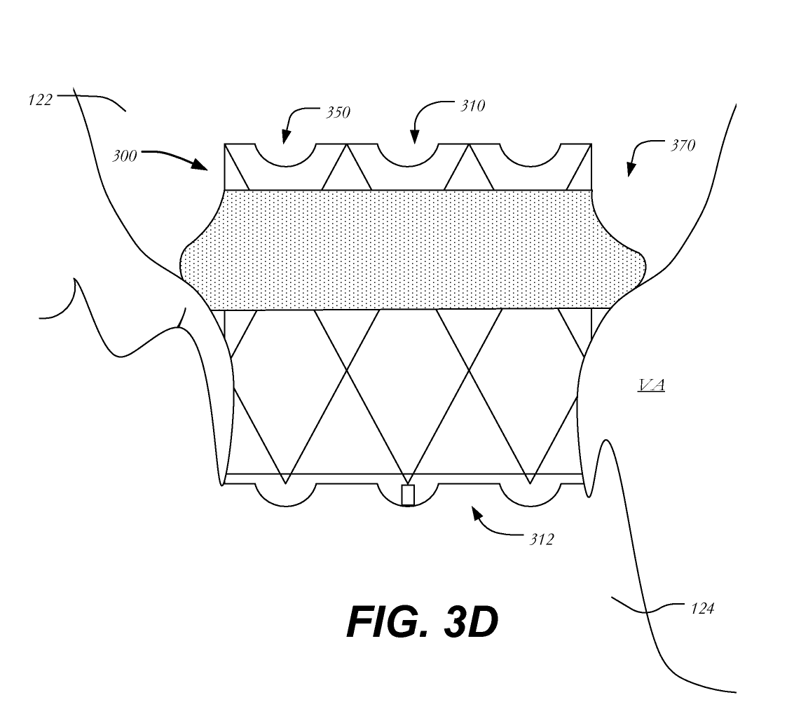

FIGS. 3A and 3B are longitudinal cross-sections of prosthetic heart valve 300 in accordance with one embodiment of the present disclosure. FIG. 3A illustrates prosthetic heart valve 300 in a relaxed configuration while FIG. 3B illustrates the prosthetic heart valve in a constrained configuration for delivery. Prosthetic heart valve 300 is a collapsible prosthetic heart valve designed to replace the function of the native mitral valve of a patient (see native mitral valve 130 of FIGS. 1-2). Generally, prosthetic valve 300 has inflow end 310 and outflow end 312. Prosthetic valve 300 may be substantially cylindrically shaped and may include features for anchoring to native heart tissue, as will be discussed in more detail below. When used to replace native mitral valve 130, prosthetic valve 300 may have a low profile so as not to interfere with atrial function in the native valve annulus.

Prosthetic heart valve 300 may include stent 350, which may be formed from biocompatible materials that are capable of self-expansion, such as, for example, shape memory alloys including Nitinol. Stent 350 may include a plurality of struts 352 that form cells 354 connected to one another in one or more annular rows around the stent. Cells 354 may all be of substantially the same size around the perimeter and along the length of stent 350. Alternatively, cells 354 near inflow end 310 may be larger than the cells near outflow end 312. Stent 350 may be expandable to provide a radial force to assist with positioning and stabilizing prosthetic heart valve 300.

Prosthetic heart valve 300 may also include valve assembly 360 including a pair of leaflets 362 attached to a cylindrical cuff 364 (best shown in FIG. 3C). Leaflets 362 replace the function of native mitral valve leaflets 136 and 138 described above with reference to FIG. 2. That is, leaflets 362 coapt with one another to function as a one-way valve. Though prosthetic heart valve 300 is illustrated as having a valve assembly 360 with two leaflets 362, it will be appreciated that prosthetic heart valve 300 may have more than two leaflets when used to replace the mitral valve or other cardiac valves within a patient. Valve assembly 360 of prosthetic heart valve 300 may be substantially cylindrical. Both cuff 364 and leaflets 362 may be wholly or partly formed of any suitable biological material, such as bovine or porcine pericardium, or polymers, such as polytetrafluoroethylene (PTFE), urethanes and the like.

When used to replace a native mitral valve, valve assembly 360 may be sized in the range of about 20 mm to about 40 mm in diameter. Valve assembly 360 may be secured to stent 350 by suturing to struts 352 or by using tissue glue, ultrasonic welding or other suitable methods.

Prosthetic heart valve 300 may further include flange 370 for anchoring the heart valve within a native valve annulus. Flange 370 may be formed of a body 372 circumferentially surrounding stent 350 and extending between attachment end 374 and free end 376. Attachment end 374 of body 372 may be coupled to selected struts 352 of stent 350 or to cuff 364 via ultrasonic welds, glue, adhesive or any other suitable means. As shown in FIGS. 3A and 3B, attachment end 374 is coupled to stent 350 near inflow end 310. In some examples, attachment end 374 may be coupled to stent 350 at a location approximately one-third of the distance from inflow end 310 to outflow end 312 or approximately halfway between inflow end 310 and valve assembly 360.

Body 372 may be formed of a braided material, in various configurations to create varying shapes and/or geometries to engage tissue. As shown in FIGS. 3A and 3B, body 372 includes a plurality of braided strands or wires 378 arranged in three-dimensional shapes. In one example, wires 378 form a braided metal fabric that is both resilient and capable of heat treatment to substantially set a desired preset shape. One class of materials which meets these qualifications is shape memory alloys. One example of a shape memory alloy is Nitinol. Wires 378 may comprise various materials other than Nitinol that have elastic and/or memory properties, such as spring stainless steel, trade named alloys such as Elgiloy®, Hastelloy®, CoCrNi alloys (e.g., trade name Phynox), MP35N®, CoCrMo alloys, or a mixture of metal and polymer fibers. Depending on the individual material selected, the strand diameter, number of strands, and pitch may be altered to achieve the desired shape and properties of flange 370.

In the simplest configuration of flange 370, shown in FIG. 3A, body 372 may be formed in a cylindrical or tubular configuration circumferentially disposed around a portion of stent 350 and/or valve assembly 360. When body 372 is formed of a shape-memory material capable of being preset, it may roll upon itself over its longitudinal axis in a furled condition to form a generally toroidal shape (best shown in FIG. 3C). After being preset, body 372 may be stretched from the furled condition (FIG. 3A) to a cylindrical unfurled condition (FIG. 3B) for loading into a delivery device and delivery into the patient. Once released from the delivery device, body 372 may return to its furled condition (e.g., return to a generally toroidal shape). Portions of body 372 may endothelialize and in-grow into the heart wall over time, providing permanent stability and a low thrombus surface. FIG. 3C is a side view illustrating body 372 of flange 370 in the furled condition. When body 372 folds upon itself in the furled condition, it radially bulges to create flange 370. In one example, flange 370 may be positioned opposite attachment end 374 of body 372. As seen from the side view, flange 370 radially extends from the outer diameter of stent 350 by a distance d1. In some examples, in the furled condition of body 372, flange 370 bulges from the outer diameter of stent 350 such that d1 is between about 4 mm to about 8 mm. Flange 370 may aid in anchoring heart valve 300 within a native valve annulus as will be described with respect to FIG. 3D.

In FIG. 3D, heart valve 300 has been implanted within native valve annulus VA between left atrium 122 and left ventricle 124. In the implanted position, body 372 is in the furled condition and forms flange 370, radially extending outwardly and disposed above native valve annulus VA. With body 372 in the furled condition, heart valve 300 is partially anchored within native valve annulus VA as flange 370 restricts heart valve 300 from slipping into left ventricle 124. Specifically, flange 370 has a diameter that is too large to pass through native valve annulus VA. Because flange 370 is coupled to stent 350, heart valve 300 is restricted from migrating into left ventricle 124 during normal operation of prosthetic heart valve 300.

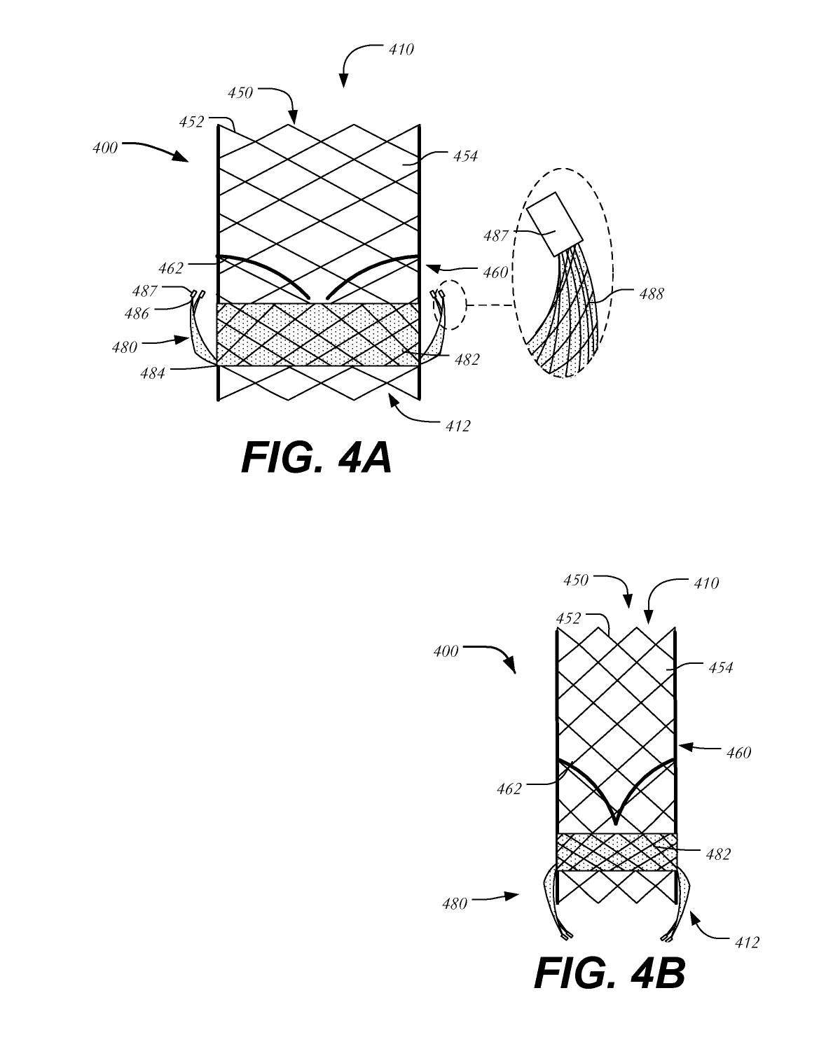

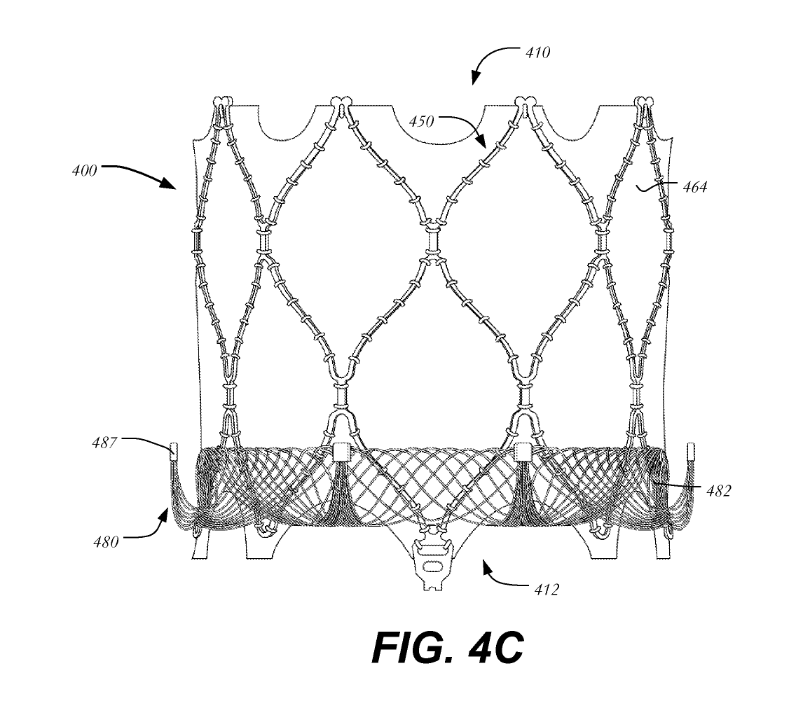

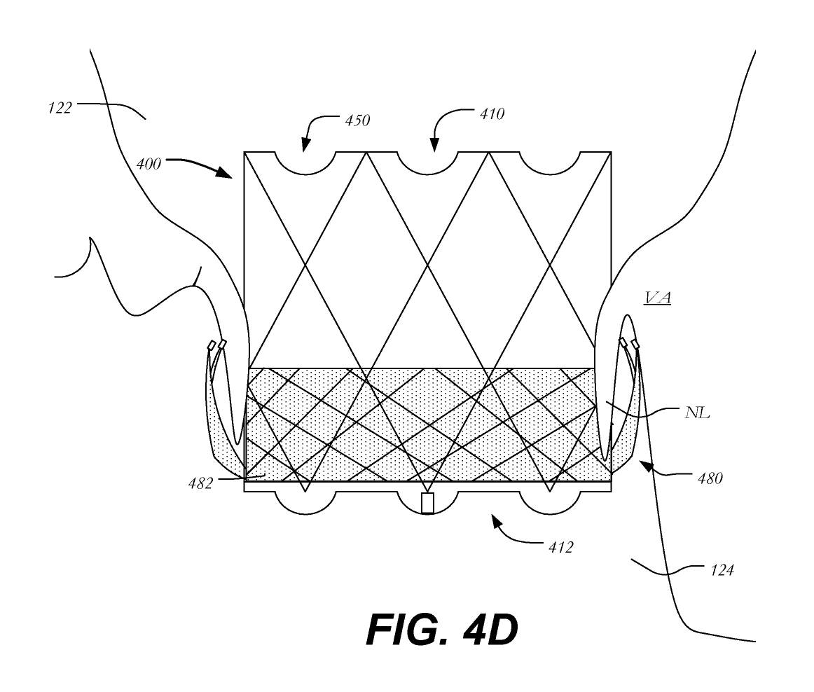

FIGS. 4A and 4B are longitudinal cross-sections of prosthetic heart valve 400 in accordance with another embodiment of the present disclosure. FIG. 4A illustrates prosthetic heart valve 400 in a relaxed configuration while FIG. 4B illustrates prosthetic heart valve in a constrained configuration for loading into a delivery device and delivery into a patient. Prosthetic heart valve 400 may extend between inflow end 410 and outflow end 412 and generally includes a substantially cylindrical stent 450 having a plurality of struts 452, which form cells 454 similar to those described above with reference to FIGS. 3A and 3B. Prosthetic heart valve 400 may further include valve assembly 460 including a pair of leaflets 462 attached to cuff 464 (see FIG. 4C).

In this embodiment, a number of hooks 480 are disposed near outflow end 412 to aid in anchoring prosthetic heart valve 400. As shown in FIGS. 4A and 4B, prosthetic heart valve 400 includes four hooks 480, disposed as two pairs of hooks 480 on contralateral ends of stent 450. It will be understood, however, that any number of hooks 480 may be provided including one, two, three, four, five, six or more hooks disposed around the circumference of stent 450. Hooks 480 may be coupled to stent 450 via ultrasonic welds, glue, adhesive or any other suitable means. In some examples, hooks 480 may be coupled to stent 450 via circumferential portion 482, which wraps around stent 450 and is attached thereto through any suitable means.

Hooks 480 may extend between attachment end 484 and free end 486 which terminates in blunt tip 487. Hooks 480 may be formed of a braided material in various configurations to create varying shapes and/or geometries to engage tissue in a manner similar to flange 370 of FIG. 3A. As shown in FIG. 4A, each hook 480 includes a plurality of braided strands or wires 488 arranged in a curved or bent three-dimensional shape. In one example, wires 488 form a braided metal fabric that is both resilient and capable of heat treatment to substantially set a desired preset shape. One class of materials which meets these qualifications is shape memory alloys. One example of a shape memory alloy is Nitinol. Wires 488 may comprise various materials other than Nitinol that have elastic and/or shape memory properties, such as spring stainless steel, trade named alloys such as Elgiloy®, Hastelloy®, CoCrNi alloys (e.g., trade name Phynox), MP35N®, CoCrMo alloys, or a mixture of metal and polymer fibers. Depending on the individual material selected, the strand diameter, number of strands, and pitch may be altered to achieve the desired properties of hook 480. Blunt tips 487 may be coupled to free ends 486 to prevent damage to the patient's tissue as will be illustrated in more detail below. Each blunt tip 487 may be in the form of a crimp tube capable of coupling wires 488 together at the free end 486 of hook 480.

When hooks 480 are formed of a shape-memory material, they may be capable of transition between two conditions, a first relaxed condition for anchoring heart valve 400 in the native valve annulus (FIG. 4A) and a second deformed condition for delivery into the patient (FIG. 4B). Specifically, after being preset (e.g., via heat setting), hooks 480 may be stretched from the first relaxed condition in which the hooks extend toward inflow end 410 to the second deformed condition in which the hooks point away from inflow end 410 for loading into a delivery device and delivery into the patient. Because hooks 480 are biased to the first condition, once released from the delivery device, hooks 480 will return to their first relaxed condition (e.g., the hooks will flip upward and point toward inflow end 410). FIG. 4C is a side view illustrating hooks 480 in the first relaxed condition. As seen in FIG. 4C, hooks 480 extend radially outward and then upward toward inflow end 410.

In FIG. 4D, heart valve 400 has been implanted within native valve annulus VA between left atrium 122 and left ventricle 124. In the implanted position, hooks 480 are in their relaxed condition, extending radially outwardly from stent 450 and then upwardly toward inflow end 410, residing between the native leaflet and the annulus. With hooks 480 in their relaxed condition, heart valve 400 is partially anchored within native valve annulus VA as hooks 480 restrict heart valve 400 from migrating into left atrium 122. Specifically, hooks 480 deploy behind native leaflets NL and anchor heart valve 400 thereto. Because hooks 480 are coupled to stent 450, heart valve 400 is restricted from migrating into left atrium 122 during normal operation of prosthetic heart valve 400.

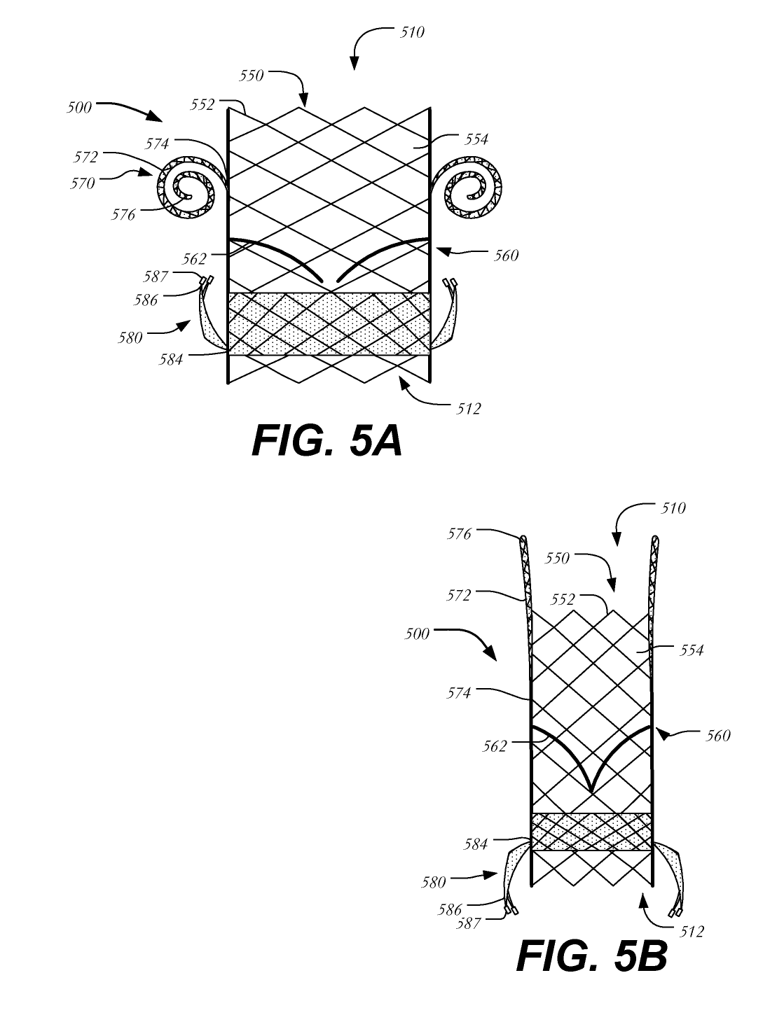

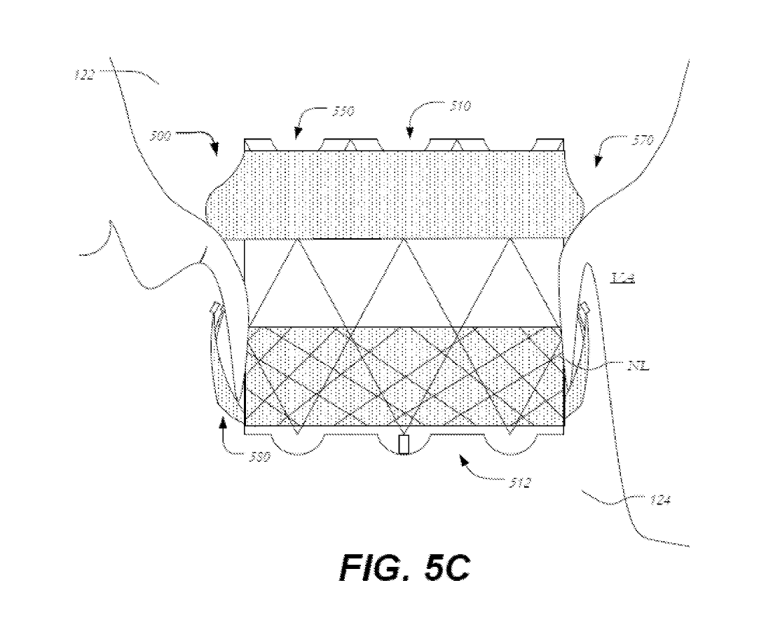

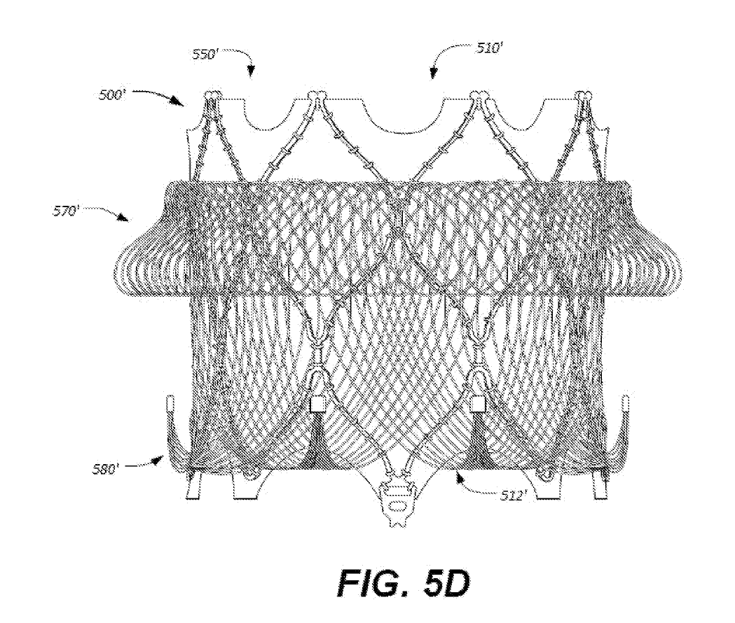

FIGS. 5A-5C illustrate another embodiment of a prosthetic heart valve with improved anchoring capability. Prosthetic heart valve 500 is a collapsible prosthetic heart valve designed to replace the function of the native mitral valve of a patient and generally has inflow end 510 and outflow end 512. Prosthetic heart valve 500 may include substantially cylindrical stent 550, which may be formed from biocompatible materials that are capable of self-expansion, such as, for example, shape memory alloys including Nitinol. Stent 550 may include a plurality of struts 552 that form cells 554 connected to one another in one or more annular rows around the stent. Prosthetic heart valve 500 may also include valve assembly 560 including a pair of leaflets 562 attached to a cylindrical cuff similar to cuff 364 shown in FIG. 3C. Both the cuff and leaflets 562 may be wholly or partly formed of any suitable material as described above with reference to cuff 364 and leaflets 362.

Similar to prosthetic heart valve 300 of FIGS. 3A-D, prosthetic heart valve 500 may further include flange 570 coupled approximately one-third of the distance from inflow end 510 to outflow end 512 or approximately halfway between inflow end 510 and valve assembly 560 for anchoring the heart valve within a native valve annulus. Flange 570 may be formed of a body 572 circumferentially surrounding stent 550 and extending between attachment end 574 and free end 576. Attachment end 574 of body 572 may be coupled to selected struts 552 of stent 550 via ultrasonic welds, glue, adhesive or any other suitable means. As shown in FIGS. 5A and 5B, attachment end 574 is coupled to stent 550 near inflow end 510. Body 572 may be formed of any of the materials described above for forming body 372 and may be capable of transitioning between an unfurled condition and a furled condition as detailed above in connection with body 372.

Prosthetic heart valve 500 also includes a number of hooks 580. In some embodiments, hooks 580 may be disposed near outflow end 512 and further away from inflow end 510 than body 572 to aid in anchoring the prosthetic heart valve. Hooks 580 may extend between attachment end 584 and free end 586 which terminates in blunt tip 587. Hooks 580 may be directly coupled to stent 550, or may be coupled to stent 550 via a circumferential portion similar to circumferential portion 482 illustrated in FIGS. 4A and 4B. In other embodiments, hooks 580 and flange 570 may be positioned similarly with respect to inflow end 510 and outflow end 512. In these embodiments, hooks 580 may be coupled to stent 550 via the hooks being coupled to body 572 which forms flange 570, such as shown in FIG. 5D. As shown in FIGS. 5A and 5B, prosthetic heart valve 500 includes four hooks 580, disposed as two pairs of hooks 580 on contralateral ends of stent 550. Hooks 580 may be formed of any of the materials described above for forming hooks 480, and may be capable of transitioning between two conditions, a first relaxed condition for anchoring heart valve 500 in the native valve annulus (FIG. 5A) and a second deformed condition for delivery into the patient (FIG. 5B). Specifically, after being preset, hooks 580 may be stretched from the relaxed condition in which the hooks extend toward inflow end 510 to the deformed condition in which the hooks point away from inflow end 510 for loading into a delivery device and delivery into the patient. Because hooks 580 are biased to the relaxed condition, they will return to their first condition once released from the delivery device (e.g., the hooks will flip upward and point toward inflow end 510). Hooks 580 are illustrated in the first relaxed condition and body 572 of flange 570 in the furled condition in FIG. 5A. As seen in FIG. 5A, hooks 580 extend radially outwardly from stent 550 and then upwardly toward inflow end 510, while body 572 bulges outward radially to form flange 570.

In FIG. 5C, heart valve 500 has been implanted within native valve annulus VA between left atrium 122 and left ventricle 124. In the implanted position, body 572 is in the furled condition and forms flange 570, radially extending above native valve annulus VA. With body 572 in the furled condition, heart valve 500 is partially anchored within native valve annulus VA as flange 570 restricts heart valve 500 from slipping into left ventricle 124. Specifically, radially expanded flange 570 is too large to pass through native valve annulus VA. Because flange 570 is coupled to stent 550, heart valve 500 is restricted from migrating into left ventricle 124 during normal operation of prosthetic heart valve 500. Likewise, hooks 580 are in their relaxed configuration, extending toward inflow end 510. In this orientation, hooks 580 deploy behind native leaflets NL and anchor heart valve 500 thereto. Because hooks 580 are coupled to stent 550, heart valve 500 is restricted from migrating into left atrium 122 during normal operation of heart valve 500. Thus, flange 570 and hooks 580 cooperate to fully anchor heart valve 500 within native valve annulus VA.

FIG. 5D illustrates an embodiment of a prosthetic heart valve 500′ that is similar to prosthetic heart valve 500 in most respects. For example, prosthetic heart valve 500′ is a collapsible prosthetic heart valve and generally has inflow end 510′, an outflow end 512′, and substantially cylindrical stent 550′, which may be formed from biocompatible materials that are capable of self-expansion, such as, for example, shape memory alloys including Nitinol. However, in this embodiment, hooks 580′ are coupled to stent 550′ via the hooks being coupled to the body that forms flange 570′. Here, flange 570′ and hooks 580′ are integrally formed.

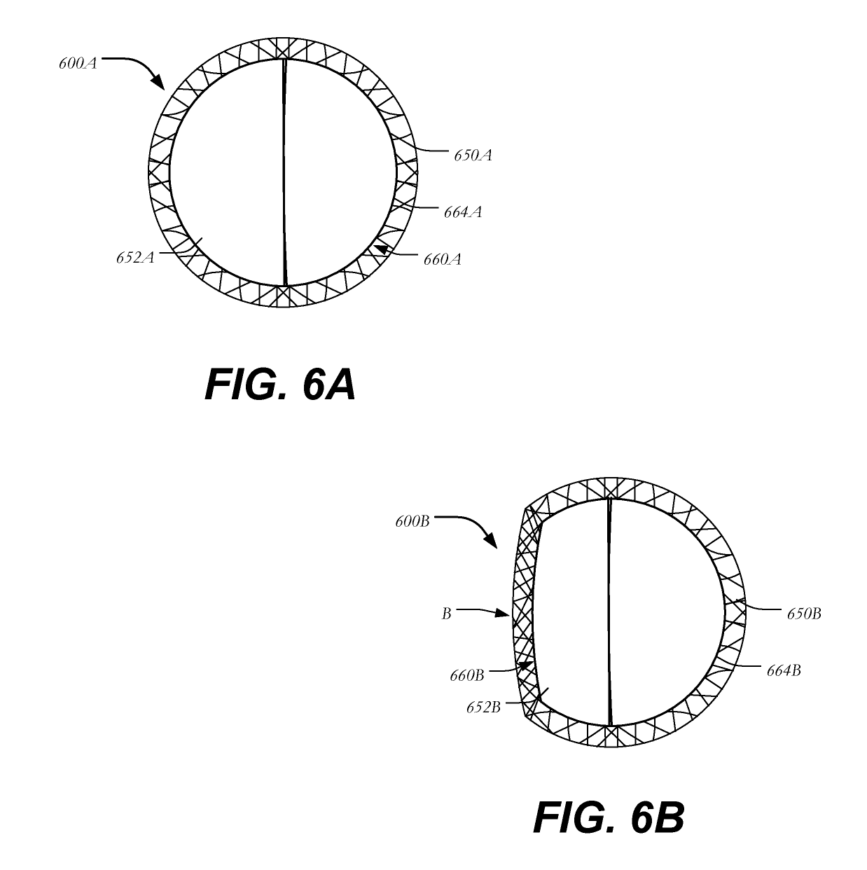

The previous embodiments have illustrated generally cylindrical prosthetic heart valves having substantially circular transverse cross-sections. FIG. 6A is a top view of one example of such a prosthetic heart valve. Specifically, prosthetic heart valve 600A includes stent 650A and valve assembly 660A including a pair of leaflets 652A attached to a cylindrical cuff 664A. As shown from this top view, stent 650A has a substantially circular transverse cross-section. Alternatively, a prosthetic heart valve may have a transverse cross-section with other shapes, such as an oval, a square, a triangle, a diamond or an irregular shape. One embodiment of a prosthetic heart valve having a non-circular transverse cross-section is shown in FIG. 6B. Prosthetic heart valve 600B includes stent 650B and valve assembly 660B including a pair of leaflets 652B attached to a cylindrical cuff 664B. As shown in FIG. 6B, stent 650B has a flattened region B to provide prosthetic heart valve 600B with a D-shaped transverse cross-section. D-shaped prosthetic heart valve 650B may be constructed to avoid impinging aortic flow. Thus, prosthetic heart valve 600B may be disposed within the native valve annulus such that region B is disposed against and/or adjacent the aorta. As seen in FIGS. 6B and 6A, leaflets 652B of heart valve 600B function in a manner similar to leaflets 652A of heart valve 600A. Leaflets 652B may be sized or shaped to accommodate flattened region B (e.g., one of the leaflets may be include a flattened portion to accommodate and attach to flattened region B).

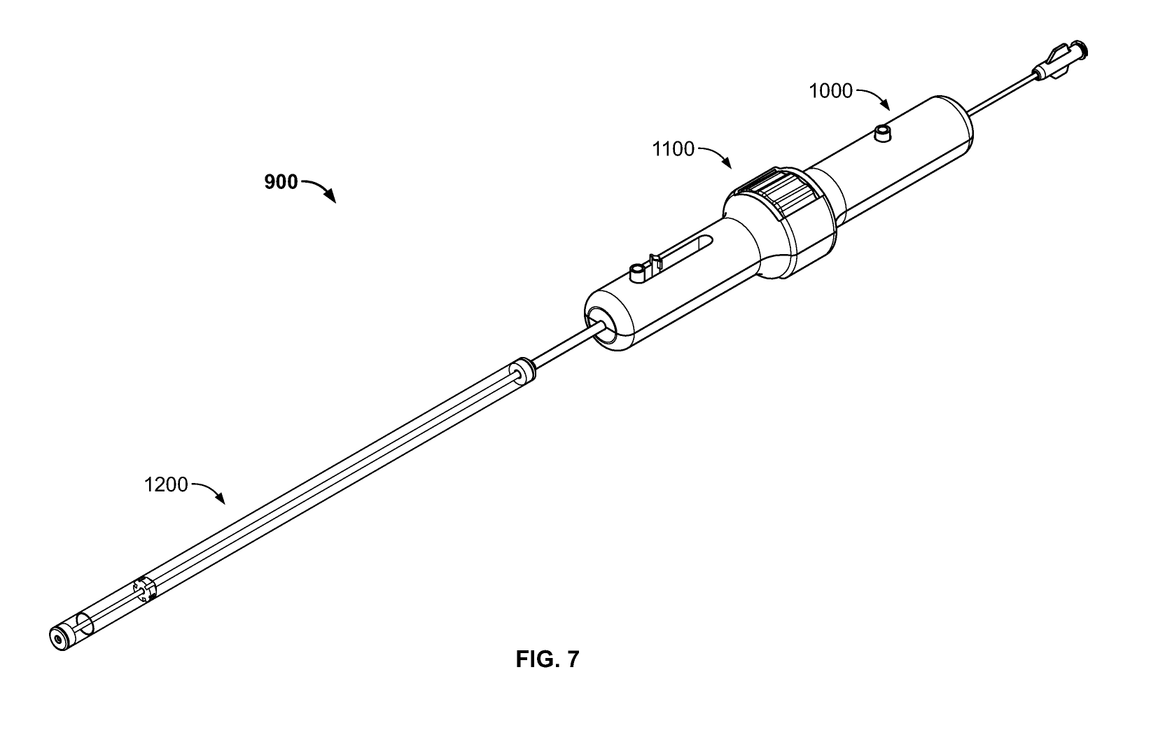

Referring now to FIG. 7, an exemplary delivery device 900 for use in delivering a collapsible prosthetic heart valve (or other types of self-expanding collapsible stents) is shown. Generally, delivery device 900 includes a handle subassembly 1000, an actuator subassembly 1100, and a catheter subassembly 1200. Catheter subassembly 1200, which is illustrated as partially transparent in FIG. 7, may function to deliver the prosthetic heart valve to and deploy the heart valve at a target location. Actuator subassembly 1100 may function to control deployment of the valve from catheter subassembly 1200. Handle subassembly 1000 may function to facilitate operation of the other components by a user. Each subassembly is described in greater detail below, followed by an exemplary method of use.

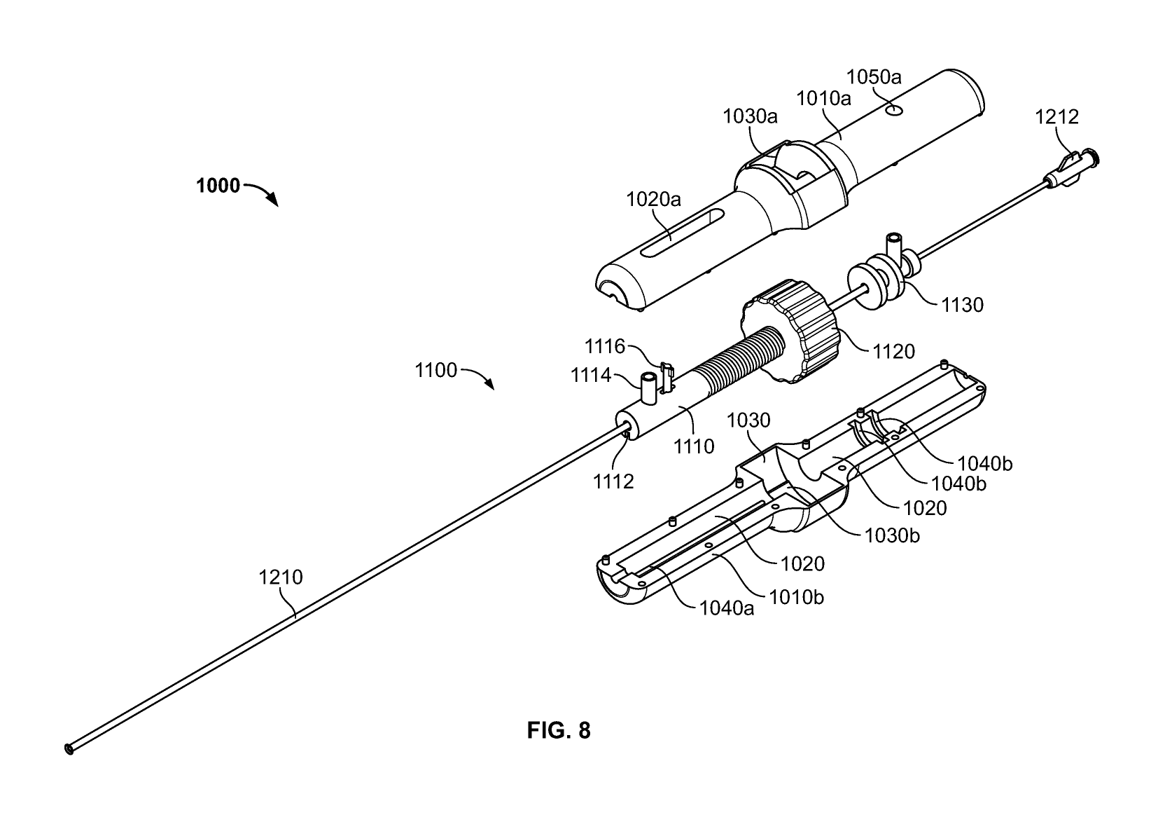

As illustrated in FIG. 8, handle subassembly 1000 includes a top portion 1010a and a bottom portion 1010b. The top and bottom portions 1010a and 1010b may be individual pieces configured to be joined to one another as shown in FIG. 8. For example, top and bottom portions 1010a and 1010b may include some combination of mating features, such as pegs and corresponding holes, to facilitate connecting the top and bottom portions together. Top and bottom portions 1010a and 1010b may be connected to one another in any other suitable manner, including, for example, by an adhesive.

Top and bottom portions 1010a and 1010b, individually or collectively, define a number of spaces to house components of actuator subassembly 1100 and catheter subassembly 1200. For example, top and bottom portions 1010a and 1010b define an elongated space 1020 in handle subassembly 1000 in which lead screw 1110 is positioned and through which the lead screw may translate. An elongated rib 1040a may be formed along the length of space 1020 in bottom portion 1010b and may be configured to mate with a corresponding groove 1112 in lead screw 1110 to guide the lead screw during translation. Top portion 1010a may also include elongated window 1020a through which a flush port 1114 and stop member 1116 may extend. Similarly, top and bottom portions 1010a and 1010b may define a generally circular or cylindrical space 1030 in which knob 1120 is positioned. Top and bottom portions 1010a and 1010b may also include top and bottom windows 1030a and 1030b, respectively, into which knob 1120 may extend such that a user may access the knob. Bottom portion 1010b may additionally include one or more semi-circular grooves 1040b to mate with corresponding flanges on flush adapter 1130. Similar grooves (not shown) may be formed in top portion 1010a. The engagement of the flanges of flush adapter 1130 in these grooves maintains the flush adapter in a fixed axial position relative to handle subassembly 1000. Finally, top portion 1010a may include flush aperture 1050a sized to receive a flush port on flush adapter 1130.

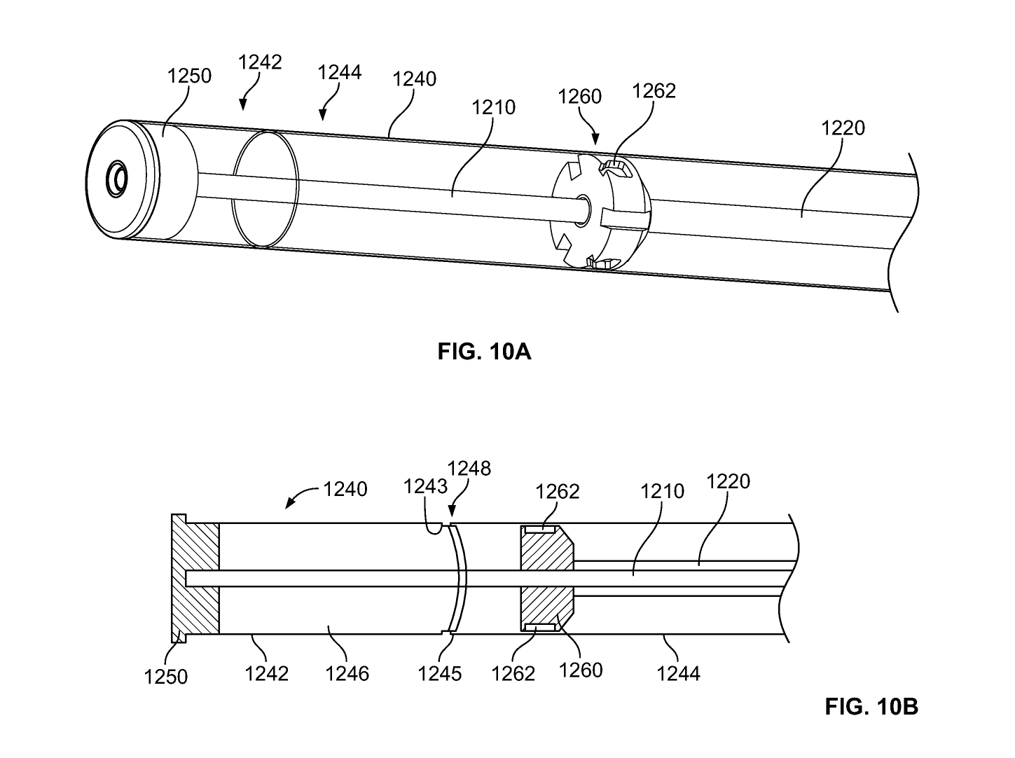

Inner core 1210 of catheter subassembly 1200 is also illustrated in FIG. 8. It should be appreciated that, although some components of catheter subassembly 1200 are illustrated in FIG. 8, others are omitted for purposes of clarity. Inner core 1210 may extend from beyond a proximal end of handle subassembly 1000, through the handle subassembly, to a distal portion of delivery device 900 (distal portion described in greater detail below with reference to FIGS. 10A-B). In particular, inner core 1210 may extend through correspondingly shaped bores through lead screw 1110 and flush adapter 1130, and to a proximal hub 1212. Proximal hub 1212 may be positioned proximally of the proximal end of handle subassembly 1000 such that, during use, a user may grip the proximal hub. The fit between inner core 1210 and the through bores of lead screw 1110 and flush adapter 1130 is preferably snug enough to keep the inner core in place until the user applies intentional force to the inner core, for example by manually pushing or pulling proximal hub 1212 distally or proximally. As noted above, when assembled, knob 1120 may be accessible through one or both of top and bottom windows 1030a and 1030b of handle subassembly 1000. Still referring to FIG. 8, knob 1120 may have a textured cylindrical surface, such as ridges, to assist the user in gripping and rotating the knob. Lead screw 1110 extends through a central aperture in knob 1120. The central aperture in knob 1120 may be internally threaded and configured to mate with external threads on lead screw 1110. As knob 1120 is longitudinally confined within windows 1030a and 1030b, rotation of knob 1120 causes lead screw 1110 to translate proximally or distally depending on the direction of rotation. The engagement of rib 1040a in groove 1112 prevents lead screw 1110 from simply rotating with knob 1120 and keeps the lead screw aligned in the longitudinal direction of handle subassembly 1000.

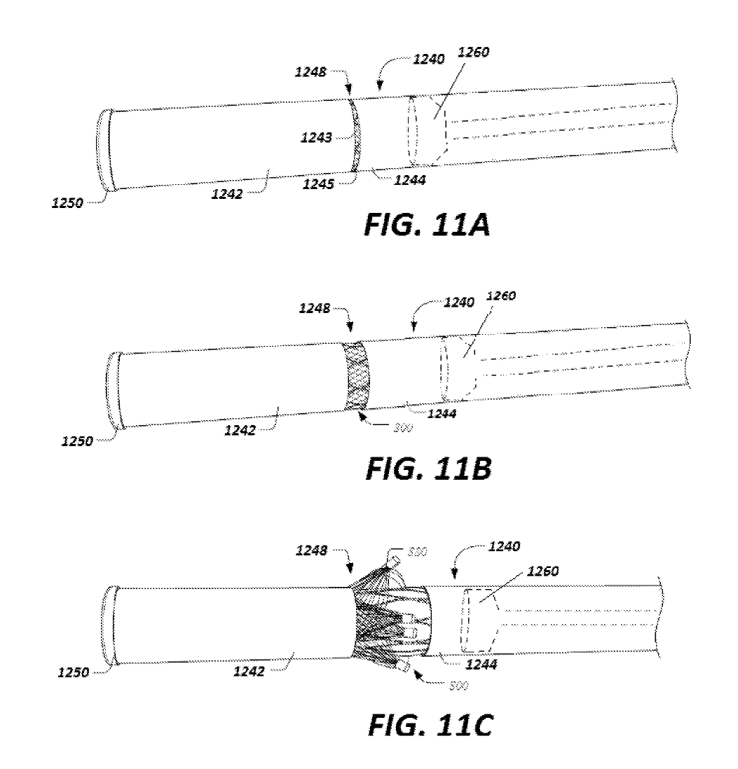

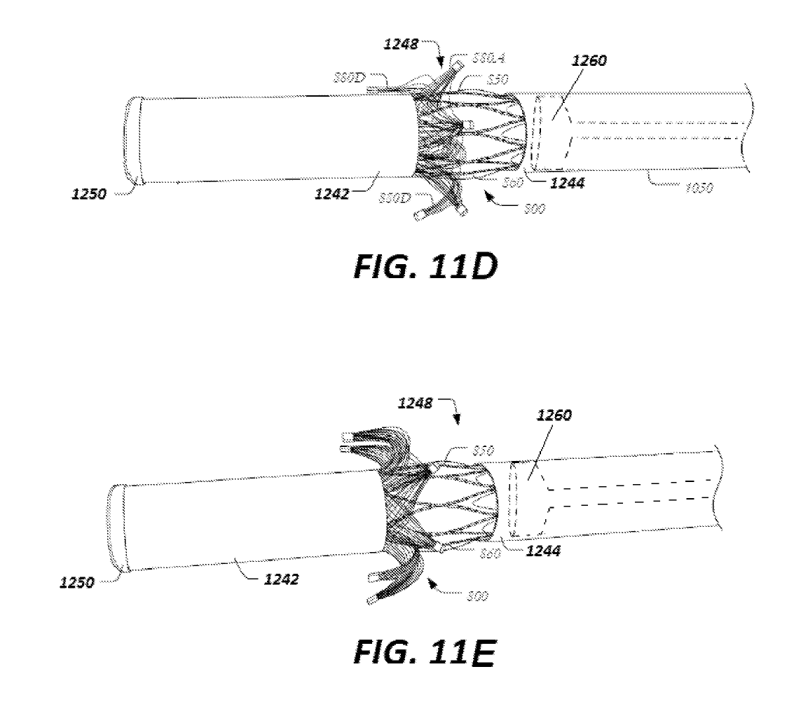

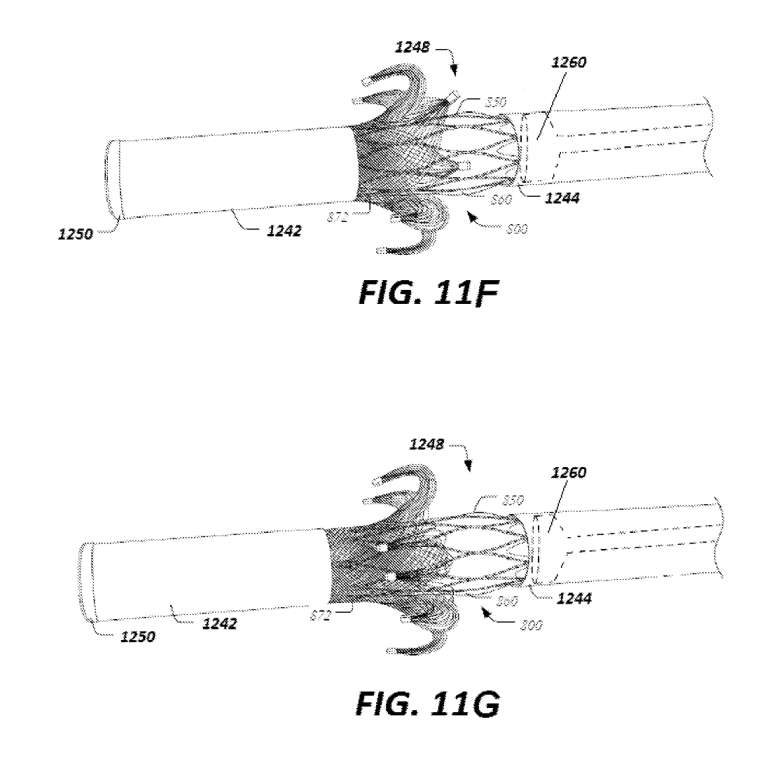

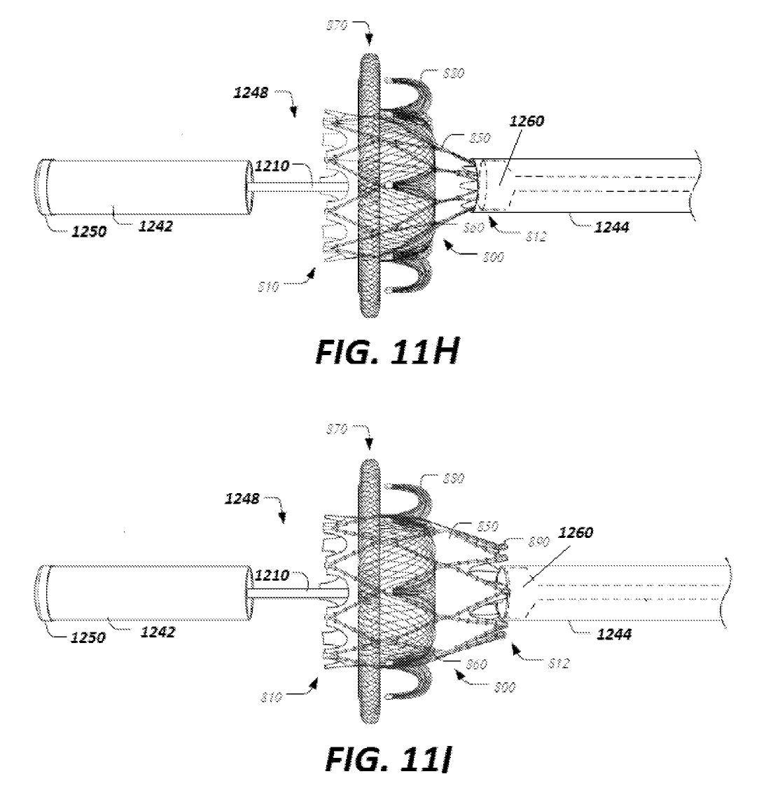

Flush port 1114 may provide fluid communication with an inside of lead screw 1110 to allow flushing of same. Flush port 1114 may further provide a limit on the distance that lead screw 1110 may translate proximally or distally. Stop member 1116, when connected to lead screw 1110, may also provide a separate limit to the proximal translation of drive screw 1110, described in greater detail below with respect to FIGS. 11A-I. One end of stop member 1116 may include a protruding tab (not shown) that, in a first rotational position, permits insertion or removal of the stop member from lead screw 1110 and, in a second rotation position, prevents insertion or removal of the stop member from the lead screw. The other end of stop member 1116 may include flanges or other structures to assist the user in grasping the stop member and rotating it from the first rotational position to the second rotational position.

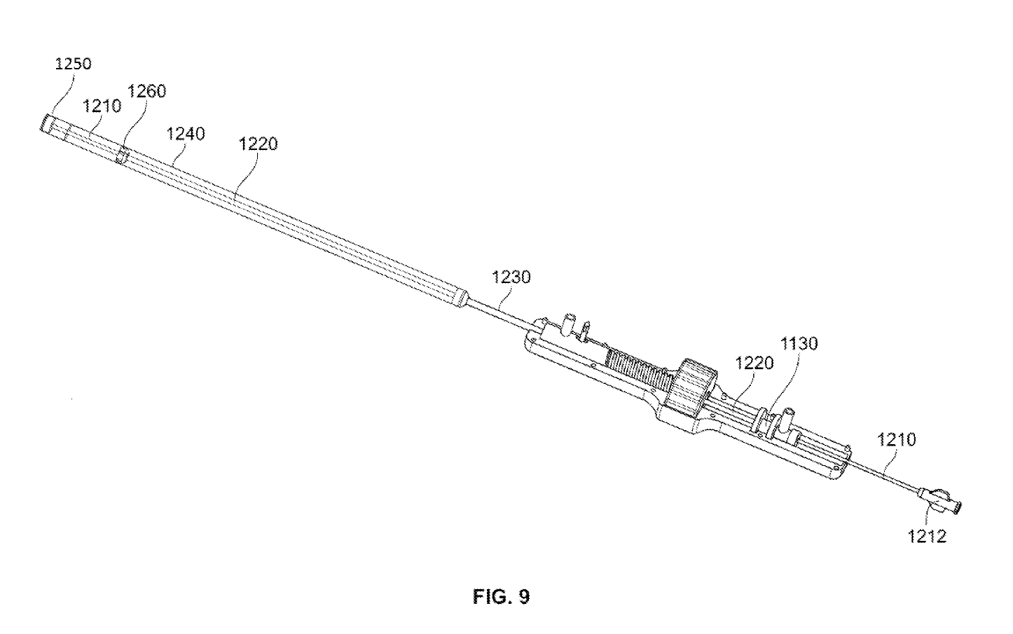

FIG. 9 best illustrates the components of catheter subassembly 1200, with top portion 1010a of handle subassembly 1000 removed. In general, catheter subassembly 1200 includes inner core 1210, described in part above in relation to FIG. 8, an inner sheath 1220, a middle sheath 1230, and an outer sheath 1240, shown in FIG. 9 as partially transparent. Inner core 1210 extends from proximal hub 1212 to an atraumatic distal tip 1250, described more fully below.

Inner sheath 1220 is positioned over inner core 1210 and extends from flush adapter 1130, through knob 1120 and lead screw 1110, and terminates at a retaining element 1260. Inner sheath 1220 is axially fixed with respect to handle subassembly 1000 due, at least in part, to its connection to flush adapter 1130, which, as described above, is held in a fixed axial position. A flush port on flush adapter 1130 provides fluid communication with the space between inner sheath 1220 and inner core 1210.

Middle sheath 1230 is positioned over inner sheath 1220 and inner core 1210, and extends from the distal end of lead screw 1110 to the proximal end of outer sheath 1240. Middle sheath 1230 is connected to both lead screw 1110 and the proximal end of outer sheath 1240 such that proximal or distal translation of lead screw 1110 causes corresponding translation of the middle sheath as well as the portion of the outer sheath to which the middle sheath is connected. In addition to enabling flushing of the interior of lead screw 1110, flush port 1114 may provide fluid communication with the space between middle sheath 1230 and inner sheath 1220 to enable the flushing of that space.

Outer sheath 1240 is positioned over inner sheath 1220 and inner core 1210, and extends from the distal end of middle sheath 1230 to atraumatic distal tip 1250. The distal portion of outer sheath 1240 is illustrated in greater detail in FIG. 10A, again with the outer sheath being illustrated as partially transparent, with a cross-sectional view shown in FIG. 10B. Distal tip 1250 may be blunt to facilitate advancement of the outer sheath without injury to the patient's tissue. For example, distal tip 1250 may have a substantially flat distal surface that is substantially perpendicular to the longitudinal axis of outer sheath 1240. This configuration may be particularly suited to use in delivering a prosthetic valve to a native mitral valve annulus. This is because, during transapical mitral valve delivery for example, there may be relatively little working space for the distal end of outer sheath 1240. If the distal end of outer sheath 1240 extends too far distally, tissue in the left atrium may be damaged. Distal tip 1250, with its atraumatic and blunted design, may help mitigate the risk of damaging native tissue during valve delivery and may maximize the working space available for outer sheath 1240. This concern is less evident during transapical aortic valve delivery where the distal end of the delivery device may be able to extend into the aortic arch, which thus provides additional working space.

As described above, inner core 1210 may be coupled to distal tip 1250. The distal end of outer sheath 1240 through which inner core 1210 extends may have a first segment 1242 and a second segment 1244. First segment 1242 may be coupled to distal tip 1250 so that movement of inner core 1210 results in a corresponding movement of first segment 1242. Second segment 1244 may be coupled to middle sheath 1230 (not visible in FIG. 10A), which in turn is coupled to lead screw 1110. First segment 1242 and second segment 1244 may include complementary coupling features such as ribs, clips or fasteners for ensuring that they do not become separated from one another during delivery of a prosthetic valve into a patient. In one example, mating end 1243 of first segment 1242 may be slightly smaller in diameter than the complementary mating end 1245 of second segment 1244 such that it may be received with a friction fit therein.

The space between inner core 1210 and the distal end of outer sheath 1240 defines a compartment 1246 for housing a prosthetic heart valve. Specifically, a prosthetic heart valve may be disposed about inner core 1210 and housed within outer sheath 1240. Compartment 1246 may be bounded at its distal end by distal tip 1250 and at its proximal end by a retaining element 1260 connected to a distal end of inner sheath 1220. Retaining element 1260 may include a plurality of receivers 1262 around its perimeter, the receivers being configured to accept retainers disposed near the outflow end of a prosthetic heart valve as will be described in more detail below. First segment 1242 and second segment 1244 of outer sheath 1240 may be translatable relative to one another to form an increasing gap 1248 therebetween so as to expose the prosthetic heart valve in compartment 1246 for deployment.

FIGS. 11A-I illustrate the process of implanting prosthetic heart valve 800 in a patient's native valve annulus using delivery device 900 described above. Only a distal portion of delivery device 900 is illustrated in FIGS. 11A-I. For the sake of clarity, the patient's tissue is not shown and retaining element 1260 (but not receivers 1262) and inner sheath 1220 are illustrated in dashed lines. In this example, prosthetic heart valve 800 extends from an inflow end 810 to an outflow end 812 and includes stent 850, valve assembly 860 and anchoring features including both a flange 870 formed of a body 872 and a plurality of hooks 880 as described above.

After loading prosthetic heart valve 800 into compartment 1246 of outer sheath 1240, first segment 1242 and second segment 1244 may be brought together to close gap 1248 and couple first mating end 1243 and second mating end 1245 together to securely enclose the prosthetic heart valve. Once first segment 1242 and second segment 1244 are secured together, closed outer sheath 1240 may be inserted into the patient and advanced to the native valve annulus for deployment of prosthetic heart valve 800 via a transapical approach. It will be understood that other delivery approaches such as transfemoral or transeptal approaches may also be possible. FIG. 11A illustrates an initial step after first segment 1242 and second segment 1244 have been disengaged from one another and gap 1248 has begun to open.

The deployment of prosthetic heart valve 800 may be accomplished in three stages. In the first stage, hooks 880 may be deployed. Flange 870 may be deployed in a second stage following the completion of the first stage. In the third stage, prosthetic heart valve 800 is fully released from delivery device 900.

To begin the first stage, second segment 1244 may be translated away from first segment 1242. This may be accomplished by pulling the portion of outer sheath 1240 that forms second segment 1244 toward handle subassembly 1000. This movement may be effected by rotating knob 1120 in a first direction to pull lead screw 1110 proximally. As lead screw 1110 translates proximally, it pulls both middle sheath 1230 and second segment 1244 proximally. At the same time, however, inner core 1210 and first segment 1242 of outer sheath 1240 remain in a fixed position relative to handle subassembly 1000, as does prosthetic heart valve 800, by virtue of the friction between inner core 1210 and flush adapter 1130. Gap 1248 may thereby enlarge to expose more of prosthetic heart valve 800 as second segment 1244 slides proximally over the prosthetic heart valve (FIG. 11B). By continuing to rotate knob 1120 to enlarge gap 1248, more of prosthetic heart valve 800 becomes exposed until the anchoring features (e.g., flange 870 and/or hooks 880) are eventually exposed. In one example, hooks 880 are exposed first, the hooks 880 being in the deformed condition extending toward the proximal end of delivery device 900. As long as the tips of hooks 880 are within second segment 1244 of outer sheath 1240 and have not yet been exposed, hooks 880 may be recaptured within second segment 1244, repositioned and redeployed.

As more of prosthetic heart valve 800 is exposed, and more specifically, as the tips of hooks 880 are exposed, the hooks begin to return to their relaxed condition (FIG. 11C). Upon emerging from outer sheath 1240, the removal of radial constraint allows hooks 880 to transition from their deformed position to their relaxed, preset position. FIG. 11D illustrates two hooks 880D that have returned to their relaxed condition (e.g., extending toward the distal end of delivery device 900) while the remaining hooks 880A remain extending toward the proximal end of delivery device 900. This process continues until the majority of hooks 880A return to their relaxed condition (FIGS. 11E and 11F). At the end of this first stage, all of hooks 880 are in the relaxed condition and extend toward the distal end of delivery device 900 (FIG. 11G).

During this first stage of release, stop member 1116 may be assembled to lead screw 1110. Stop member 1116 may be spaced in relation to the elongated window 1020a of the top portion 1010a of handle subassembly 1000 such that, as the user rotates knob 1120 and both lead screw 1110 and the stop member move proximally through the elongated window, hooks 880 become exposed just prior to the stop member making contact with the proximal end of the elongated window. Thus, while stop member 1116 is assembled to lead screw 1110, the user may rotate knob 1120 to move outer sheath 1240 proximally only until hooks 880 are released, but not farther than that. This feature helps ensure that prosthetic valve 800 is not unintentionally released from its connection with retaining element 1260 earlier than intended. Also, by staging delivery to release hooks 880 first, the user can position the hooks on the native leaflets NL, as illustrated in FIGS. 4D and 5C, before continuing the release of prosthetic heart valve 800.

In the second stage of deployment, flange 870 will form to provide a second anchoring feature. Specifically, body 872, which will form flange 870, is partially exposed in its unfurled condition (FIG. 11G). Once body 872 is fully exposed (i.e., removed from radial constraint by outer sheath 1240), the shape memory material of body 872 will return the body to its furled condition to form flange 870 (FIG. 11H). This second stage of release may be effected by translating first segment 1242 of outer sheath 1240 away from second segment 1244. The user may accomplish this movement by pushing inner core 1210 distally, for example by gripping proximal hub 1212, which extends proximally of handle subassembly 1000, and manually pushing it distally with sufficient force to overcome the friction between inner core 1210 and flush adapter 1130. It should be appreciated that other mechanisms may be used as well, for example a second knob may be used with an additional lead screw within handle subassembly 1000 to control motion of inner core 1210 in a manner similar to that in which knob 1120 controls the motion of middle sheath 1230. During this distal pushing of inner core 1210, stop member 1116 is at a proximal most position and in contact with the proximal end of elongated window 1020a. During this distal pushing of inner core 1210, gap 1248 may continue to enlarge and to expose more of prosthetic heart valve 800 as first segment 1242 slides distally over the prosthetic heart valve. As seen in FIG. 11H, outflow end 812 of prosthetic heart valve 800 remains disposed within second segment 1244 of outer sheath 1240 and coupled to retaining element 1260 while inflow end 810 is deployed subsequent to flange 870. As noted above, retaining element 1260 may include a plurality of receivers 1262 that accept retainers 890 (best shown in FIG. 11I) disposed on the outflow end 812 of prosthetic heart valve 800. The retention of retainers 890 in receivers 1262 prevents outflow end 812 of prosthetic valve 800 from being inadvertently or unintentionally deployed from second segment 1244 of outer sheath 1240.

In the third stage of deployment, prosthetic heart valve 800 is released from delivery device 900 in its entirety. To release prosthetic heart valve 800, second segment 1244 is once again translated away from first segment 1242. To accomplish this, stop member 1116 is first rotated from its original rotational position in which a protruding tab locks the stop member to lead screw 1110, to a second rotational position. In the second rotational position, the protruding tab aligns with the aperture in lead screw 1110, allowing its removal from the lead screw. Once stop member 1116 is removed, lead screw 1110 is free to translate farther proximally upon further rotation of knob 1120. As the user continues to rotate knob 1120 in the first direction, lead screw 1110, as well as middle sheath 1230 and the portion of outer sheath 1240 forming second segment 1244, continue to translate proximally relative to handle subassembly 1000 and to retaining element 1260. Translation of second segment 1244 proximally in relation to retaining element 1260 exposes retainers 890 and allows them to disengage from receivers 1262 of retaining element 1260. Once disengaged from retaining element 1260, prosthetic heart valve 800 may fully deploy. When prosthetic heart valve 800 has been fully deployed, delivery device 900 may be pulled through the interior of the deployed heart valve and removed from the patient's body.

While a three-stage deployment method for transapical delivery of a prosthetic heart valve 800 with hooks 880 and a flange 870 has been described above, a person of skill in the art would understand variations that may be made to the deployment process, particularly for different delivery routes and different prosthetic valves. For example, transapical delivery of a prosthetic valve having hooks but no flange may have a substantially similar deployment as described above, with hooks being released first, an inflow end being released second, and the valve being fully released in a third step. Similarly, transapical delivery of a prosthetic valve having a flange but no hooks may have a substantially similar deployment as described above, with an outflow end being released first, the flange being released second, and the valve being fully released in a third step. It should also be understood that, while the outflow end of a prosthetic mitral valve is generally at least partially released prior to the inflow end in order to first position the valve assembly in the native valve annulus, a user may first release the inflow end and/or flange first if desired. It should further be understood that, when using other delivery routes, such as a transfemoral route, the three-stage deployment may be modified. For example, with a transfemoral delivery, a distal portion of the delivery device may be advanced to first release the hooks, then a proximal portion retracted to release the flange and then further retracted to fully release the prosthetic valve. Methods employing such variations are within the scope of this disclosure.

It will be appreciated that the various dependent claims and the features set forth therein can be combined in different ways than presented in the initial claims. For example, any combination of flanges or hooks may be combined in a prosthetic heart valve. Additionally, it will be understood that while a transapical delivery approach has been described, the present disclosure contemplates the use of transeptal delivery as well as less conventional approaches, such as direct access to the left atrium or access into the left atrium via the left arterial appendage or the pulmonary veins. It is also conceivable that the device may be delivered by passing through the femoral artery, the aortic valve and the left ventricle. It will be appreciated that any of the features described in connection with individual embodiments may be shared with others of the described embodiments.

In embodiments according to the disclosure, a prosthetic heart valve having an inflow end and an outflow end may include a stent having a collapsed condition and an expanded condition, a collapsible and expandable valve assembly disposed within the stent and having a plurality of leaflets, and a body coupled to the stent and formed of braided wire, the body being transitionable between an unfurled condition and a furled condition, the furled condition forming a flange capable of at least partially anchoring the prosthetic heart valve within a native valve annulus; and/or

the flange may have a transverse cross-section greater than a transverse cross-section of the stent in the expanded condition for at least partially anchoring the prosthetic heart valve within the native valve annulus; and/or

the flange may be formed adjacent the inflow end of the prosthetic heart valve; and/or

the body may include a shape-memory material such as braided Nitinol; and/or

the body may promote endothelialization; and/or

the prosthetic heart valve may be loadable in a catheter with the body in the unfurled condition, the body being preset to return to the furled condition when deployed from the catheter; and/or

the prosthetic heart valve may be configured to replace a native mitral valve; and/or

the valve assembly may include two leaflets; and/or

the heart valve may further include a plurality of hooks coupled to the stent and formed of braided wire, the plurality of hooks being transitionable between a deformed condition and relaxed condition, the plurality of hooks extending toward the inflow end in the relaxed condition to at least partially anchor the prosthetic heart valve within a native valve annulus.

In other embodiments according to the disclosure, a prosthetic heart valve having an inflow end and an outflow end may include a stent having a collapsed condition and an expanded condition, a collapsible and expandable valve assembly disposed within the stent and having a plurality of leaflets, and a plurality of hooks coupled to the stent and transitionable between a deformed condition and a relaxed condition, the plurality of hooks extending toward the inflow end in the relaxed condition to at least partially anchor the prosthetic heart valve within a native valve annulus; and/or

the plurality of hooks may be formed adjacent the outflow end of the prosthetic heart valve; and/or

the plurality of hooks may include a shape-memory material such as braided Nitinol; and/or

the prosthetic heart valve may be loadable in a catheter with the plurality of hooks in the deformed condition, the plurality of hooks being preset to return to the relaxed condition when deployed from the catheter; and/or

the heart valve may further include a body coupled to the stent and formed of braided wire, the body being transitionable between an unfurled condition and a furled condition, the furled condition forming a flange capable of at least partially anchoring the prosthetic heart valve within a native valve annulus; and/or

the stent may have a non-circular transverse cross-section; and/or

the stent may have a D-shaped transverse cross-section.

In still other embodiments according to the disclosure, a method of deploying a prosthetic heart valve from a delivery device at a target site in a patient, the heart valve including a stent having a collapsed condition and an expanded condition, a collapsible and expandable valve assembly disposed within the stent, a body assembled to the stent and transitionable between a furled condition and an unfurled condition, and a plurality of hooks coupled to the stent and being transitionable between a deformed condition and a relaxed condition, may include (i) introducing the prosthetic heart valve to the target site in a collapsed condition; (ii) deploying the plurality of hooks to transition the plurality of hooks from the deformed condition to the relaxed condition; (iii) deploying the body to transition the body from the unfurled condition to the furled condition; and (iv) decoupling the prosthetic heart valve from the delivery device after the plurality of hooks are in the relaxed condition and the body is in the furled condition, whereby the plurality of hooks and the body cooperate to anchor the prosthetic heart valve at the target site. The target site may be the mitral valve annulus of the patient.

In yet another embodiment according to the disclosure, a delivery device for a collapsible medical device may include a handle and a catheter member extending from the handle and having a first portion, a second portion, and a compartment for receiving the medical device, the first portion being operably coupled to a first shaft that is axially translatable with respect to the handle and the second portion being operably coupled to a second shaft that is axially translatable with respect to the handle and to the first shaft; and/or

a distal end of the first portion may include a tip having a distal surface that is substantially perpendicular to a longitudinal axis of the first portion; and/or

the second portion may include a retaining element for retaining the medical device during deployment of the medical device; and/or

the first and second portions may have complementary coupling features; and/or

the first portion may have a first mating end with a first diameter and the second portion may have a second mating end with a second diameter, the first diameter being different than the second diameter; and/or

the handle may have an actuation member and the second shaft may be operably coupled to the actuation member; and/or

manipulation of the actuation member may cause axial movement of the second shaft; and/or

the delivery device may also include a stop member removably coupled to the second shaft, wherein, when the stop member is coupled to the second shaft, the second shaft may be capable of a first amount of axial movement and when the stop member is not coupled to the second shaft, the second shaft may be capable of a second amount of axial movement greater than the first amount.

In still a further embodiment according to the disclosure, a method of delivering a medical device into a patient may include (a) providing a delivery device including a handle and a catheter member extending from the handle and having a first portion with a first shaft operably coupled thereto, a second portion with a second shaft operably coupled thereto, and a compartment, the medical device being positioned in the compartment; (b) advancing the catheter member to an implant site within the patient; (c) axially translating the second shaft in a first axial direction; (d) axially translating the first shaft in a second axial direction opposite the first axial direction; and (e) further axially translating the second shaft in the first axial direction; whereby each axial translation in steps (c) through (e) is performed in sequence and each sequential axial translation at least partially releases the medical device from the compartment; and/or

the medical device may include a first anchoring feature, a second anchoring feature, and a retaining feature, wherein the axial translation in step (c) may release the first anchoring feature from the compartment; and/or

the axial translation in step (d) may release the second anchoring feature from the compartment; and/or

the axial translation in step (e) may release the retaining feature from the compartment; and/or

the axial translation of the second shaft in the first axial direction may be limited to a first distance while a stop member is coupled to the second shaft; and/or

the second shaft may be capable of translation in the first axial direction a total distance greater than the first distance when the stop member is not coupled to the second shaft; and/or

the axial translation in step (d) may include sliding the first shaft in the second axial direction through the first shaft.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. It will be appreciated that the various dependent claims and the features set forth therein can be combined in different ways than presented in the initial claims. It will also be appreciated that the features described in connection with individual embodiments may be shared with others of the described embodiments.

1. A prosthetic heart valve having an inflow end and an outflow end, comprising:
a stent having a collapsed condition and an expanded condition;
a collapsible and expandable valve assembly disposed within the stent and having a plurality of leaflets; and
a body coupled to the stent and formed of braided wire, the body being transitionable between an unfurled condition and a furled condition, the furled condition forming a flange capable of at least partially anchoring the prosthetic heart valve within a native valve annulus.

2. The prosthetic heart valve of claim 1, wherein the flange has a transverse cross-section greater than a transverse cross-section of the stent in the expanded condition for at least partially anchoring the prosthetic heart valve within the native valve annulus.

3. The prosthetic heart valve of claim 1, wherein the flange is formed adjacent the inflow end of the prosthetic heart valve.

4. The prosthetic heart valve of claim 1, wherein the body comprises a shape-memory material.

5. The prosthetic heart valve of claim 1, wherein the body comprises braided Nitinol.

6. The prosthetic heart valve of claim 1, wherein the body promotes endothelialization.

7. The prosthetic heart valve of claim 1, wherein the prosthetic heart valve is loadable in a catheter with the body in the unfurled condition, the body being preset to return to the furled condition when deployed from the catheter.

8. The prosthetic heart valve of claim 1, wherein the prosthetic heart valve is configured to replace a native mitral valve.

9. The prosthetic heart valve of claim 1, wherein the valve assembly includes two leaflets.

10. The prosthetic heart valve of claim 1, further comprising a plurality of hooks coupled to the stent and formed of braided wire, the plurality of hooks being transitionable between a deformed condition and relaxed condition, the plurality of hooks extending toward the inflow end in the relaxed condition to at least partially anchor the prosthetic heart valve within a native valve annulus.

11. A prosthetic heart valve having an inflow end and an outflow end, comprising:
a stent having a collapsed condition and an expanded condition;
a collapsible and expandable valve assembly disposed within the stent and having a plurality of leaflets; and
a plurality of hooks coupled to the stent and transitionable between a deformed condition and a relaxed condition, the plurality of hooks extending toward the inflow end in the relaxed condition to at least partially anchor the prosthetic heart valve within a native valve annulus.

12. The prosthetic heart valve of claim 11, wherein the plurality of hooks are formed adjacent the outflow end of the prosthetic heart valve.

13. The prosthetic heart valve of claim 11, wherein the plurality of hooks comprise a shape-memory material.

14. The prosthetic heart valve of claim 11, wherein the plurality of hooks comprise braided Nitinol.

15. The prosthetic heart valve of claim 11, wherein the prosthetic heart valve is loadable in a catheter with the plurality of hooks in the deformed condition, the plurality of hooks being preset to return to the relaxed condition when deployed from the catheter.

16. The prosthetic heart valve of claim 11, further comprising a body coupled to the stent and formed of braided wire, the body being transitionable between an unfurled condition and a furled condition, the furled condition forming a flange capable of at least partially anchoring the prosthetic heart valve within a native valve annulus.

17. The prosthetic heart valve of claim 11, wherein the stent has a non-circular transverse cross-section.

18. The prosthetic heart valve of claim 11, wherein the stent has a D-shaped transverse cross-section.

19. A method of deploying a prosthetic heart valve from a delivery device at a target site in a patient, the heart valve including a stent having a collapsed condition and an expanded condition, a collapsible and expandable valve assembly disposed within the stent, a body assembled to the stent and being transitionable between an unfurled condition and a furled condition, and a plurality of hooks coupled to the stent and being transitionable between a deformed condition and a relaxed condition, the method comprising:
introducing the prosthetic heart valve to the target site in a collapsed configuration;
deploying the plurality of hooks to transition the plurality of hooks from the deformed condition to the relaxed condition;
deploying the body to transition the body from the unfurled condition to the furled condition; and
decoupling the prosthetic heart valve from the delivery device after the plurality of hooks are in the relaxed condition and the body is in the furled condition, whereby the plurality of hooks and the body cooperate to anchor the prosthetic heart valve at the target site.

20. The method of claim 19, wherein the target site is the mitral valve annulus of the patient.
